(19) United States
(12) Patent Application Publication
(10) Pub. No.: US 2010/0041876 A1
(43) Pub. Date: Feb. 18, 2010

(54) PROCESS FOR PREPARING AROMATIC DIAZONIUM SALTS

(75) Inventors: Bernd Schmidt, Potsdam (DE); Frank Holter, Potsdam (DE)

Correspondence Address: DARBY & DARBY P.C., P.O. BOX 770, Church Street Station, New York, NY, 10008-0770, US

(73) Assignee: ZYLUM BETEILIGUNGSGESELLSCHAFT MBH & CO. PATENTE II KG, SCHONEFELD/WALTERSDORF (DE)

(21) Appl. No.: 12/513,965

(22) PCT Filed: Nov. 7, 2007
(86) PCT No.: PCT/EP07/61999

§ 371 Date: Oct. 28, 2009

(30) Foreign Application Priority Data
Nov. 8, 2006 (DE) 10 2006 053 064.0

(51) Int. Cl. C07C 245/20

(52) U.S. Cl. 534/559; 534/565

(57) ABSTRACT

A process is described for preparing aromatic diazonium salts, wherein the starting compound is an aromatic amide and the amide bond is first split hydrolytically and the amine compound thus obtained is diazotized with an inorganic nitrite salt. The diazonium salts thus obtained can be converted to stable salts and then serve as starting materials for further reactions, such as the Heck reaction.

The present invention relates to a process for the synthesis of aromatic diazonium salts.

Aromatic diazonium salts are known to be very reactive arylating reagents. However, the isolation of these salts is difficult, because they are re-active and unstable compounds, which tend to decompose.

It is a further disadvantage that these compounds are synthesized from amines. Amines themselves are reactive and, under normal conditions, already tend to decompose and, especially, to oxidize. For this reason, the amines used must be of sufficient purity, in order to decrease the formation of undesirable byproducts and impurities.

It is therefore an object of the present invention to make available a process, which overcomes the disadvantages of the prior art, for the synthesis of aromatic diazonium salts.

According to the invention, this object is accomplished by a process for the synthesis of aromatic diazonium salts, wherein the starting compound is an aromatic amide, the amide bond initially is split hydrolytically and the amine compound, so obtained, is diazotized with an inorganic nitrite salt.

According to the invention, the diazonium salt obtained preferably is stabilized with a complex anion and isolated.

Furthermore, according to the invention, further reactive groups, which may be present at the aromatic amide, preferably are previously provided with protective groups, which are not split off under the conditions of splitting the amide hydrolytically.

According to the invention, the hydrolytic splitting preferably is carried out with a mineral acid.

In this connection, it is particularly preferred if the hydrolytic splitting is carried out with an alcoholic mineral acid, the alcohol being a C₁-C₄ alcohol. Moreover, it is furthermore particularly preferred if the mineral acid is hydrochloric acid, hydrobromic acid or sulfuric acid. Moreover, it is also particularly preferred if the alcohol is methanol, ethanol, n-propanol or isopropanol. Butanols, such as 1-butanol, 2-butanol, 2-methyl-i-propanol and 2-methyl-2-propanol, are also suitable alcohols.

Furthermore, a process, for which the hydrolytic splitting of the amide is carried out at a temperature between 20° C. and 100° C., is preferred according to the invention.

Furthermore, a process, for which the diazotization is carried out at a temperature between −10° C. and +10° C., is preferred according to the invention.

Particularly preferred according to the invention is a process, for which the hydrolytic splitting of the amide and the diazotization are carried out within the same reaction mixture, without isolating the amine. The inventive process is based on a one-vessel reaction. This has the advantage of reducing the number of synthesis steps and thus leads to saving time and resources.

Furthermore, the diazonium salt obtained preferably is converted into a stable salt and optionally isolated.

A further object of the present invention moreover is a process for the synthesis of p-benzyloxyphenyldiazonium tetrafluoroborte, wherein 4-acetamido-phenol is reacted in a known manner with benzyl bromide and protected, the amide bond of the product, so obtained, is split selectively with aqueous, alcoholic mineral acid and the mixture, so obtained, is then diazotized in a known manner with an organic nitrite salt and, optionally, the diazonium salt obtained is converted into the tetrafluoroborate salt with NH₄BF₄ and optionally isolated.

The inventive process has a series of advantages. The aromatic amides, used as starting materials, can generally be synthesized easily and, as stable compounds, can be purified well or synthesized with a high degree of purity.

Aromatic amides, in the sense of the present invention, are understood to be compounds of the general Formula I

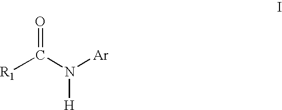

in which
R¹ is a linear or branched aliphatic C₁ to C₆ group and
Ar is an aryl, alkaryl, heteroaryl or heteroalkylaryl group, which optionally is substituted with further functional groups.

Methyl, ethyl, propyl, isopropyl, butyl, isobutyl, t-butyl, pentyl, isopentyl, hexyl, isohexyl and cyclohexyl are particularly suitable R₁ groups.

Particularly suitable Ar groups are derived from benzene, toluene, xylylene, pyrazolene, imidazole, oxazolone, thiazole, triazole, pyridine, pyridazine, pyrimidine, pyrazine, naphthalene, purine, petridine, quinoline, isoquinoline and anthracene, which may also be substituted with aliphatic C₁ to C₄ groups.

The aromatic amides, which are used according to the invention as starting materials, accordingly are amides, which are formed from aromatic amines and aliphatic acids.

The starting compounds particularly advantageously may have further functional groups. Such groups may, for example, be hydroxy groups, thiol groups, carboxyl groups, amide groups, carboxamide groups, nitrile groups or imino groups, but are not limited to these groups. Optionally these groups must be protected by means of protective groups. The protective groups are to be selected in such a way that they withstand, on the one hand, the hydrolytic splitting of the amide group and on the other, the diazotization reaction. Such protective groups are known to those of ordinary skill in the art and are described in “Protective Groups in Organic Chemistry” by Theodora W. Greene, published by Wiley in 1981.

The end products of the inventive process, the corresponding aromatic diazonium salts, are reactive starting materials, which can be converted by known reactions, of which the Japp-Klingemann reaction, deamination, the Sandmeyer reaction, the Schiemann reaction, the Meerwein reaction and the Gomberg reaction are examples. In particular, the diazonium salts, synthesized according to the invention, are suitable for the Heck reaction.

The reactions named and further reactions are suitable, particularly, for the synthesis of active pharmaceutical ingredients. The aromatic diazonium salts namely have a high purity or can be purified easily, so that, in the course of further reactions, lesser amounts of by-products and, with that, of impurities are formed. This is a significant advantage over diazotization reactions of the prior art.

The following examples explain the invention, but are not intended to limit it.

### EXAMPLES

### Example 1

### General Procedure for the Synthesis of Aromatic Diazonium Salts

The aromatic amide (0.1 moles), which optionally was provided previously with protective groups for protecting further reactive groups, is dissolved or suspended in half concentrated mineral acid (1 mole) and alcohol (C₁-C₄, 100 mL) is added. The mixture is refluxed for 1 to 5 hours and then cooled to below 10° C. An aqueous solution of sodium nitrite (1 mole) is then added dropwise. During this addition, the temperature of the reaction mixture must not exceed the starting value. The solution obtained can then be processed further in the usual manner.

### Example 2

Synthesis of p-benzyloxyphenyldiazonium Acetamide
a) N-(4-benzyloxyphenyl)diazonium Tetraborate

Potassium carbonate (6.60 g, 0.048 moles) is added to a solution of 4-acetamidophenol (6.00 g, 0.040 moles) and benzyl bromide (5.2 mL, 0.044 moles) in acetone (100 mL). The mixture is refluxed for 4 hours, cooled to ambient temperature and filtered. The precipitate is washed with acetone (100 mL), the combined filtrates are concentrated and the title compound is obtained (9.29 g, 97%), which can be used in the next step without further purification.

—H—NMR (400 MHz, CDCl₃): δ=7.43−7.30 (m, 8H, Ar+NH), 6.92 (d, J=8,9 Hz, 2H), 5.03 (s, 2H, —OCH₂—), 2.13 (s, 3H, —CH₃). ¹³C—NMR (125 MHz, CDCl₃): δ=168.3 (0), 155.5 (0), 136.9 (0), 131.2 (0), 128.5 (1), 127.9 (1), 127.4 (1) , 121,8 (1), 115.1 (1), 70.2 (2), 24.3 (3).

b) p-Benzyloxyphenyldiazonium Tetrafluoroborate

A suspension of N-(4-benzyloxy-phenyl) acetamide (5.80 g, 0.024 moles) in hydrochloric acid (3 moles/L, 80 mL) and methanol (20 mL) is refluxed for three hours. The resulting solution is cooled to 0° C. and a solution of sodium nitrite (1.66 g, 0.024 moles) in water (2 mL) is added dropwise. After stirring for one hour at this temperature, NH₄BF₄ (2.77 g, 0.026 moles) is added in small portions and the resulting suspension is stirred for 30 minutes at 0° C. The precipitate is filtered off, washed with cold water (100 mL), ethanol (100 mL) and diethyl ether (100 mL) and yields the title compound (2.70 g, 34%).

### ¹H—NMR (400 MHz, DMSO-d₆): δ=8.62 (d, J=9.4 Hz, 2H), 7.56 (d, J=9.4 Hz, 2H), 7.49 (d, J=7.4 Hz, 2H, -Ph), 7.51−7.39 (3H, -Ph), 5.42 (s, 2H, —OCH₂—).

¹³C—NMR(125 MHz, DMSO-d₆): δ=167.8 (0), 136.2 (1), 134.9 (humans), 128.7 (1), 128.4 (1), 117.9 (1), 103.7 (0), 71.4 (2).

Elementary analysis for C₁₃H₁₁BF₄N₂O.

Calc. C, 52.4; H, 3.7; N, 9.4. Found: C, 52.6; H 3.6; N, 9.6.

1. A process for synthesizing aromatic diazonium salts, which comprises hydrolyzing an aromatic amide, to form an amine compound and dizaotizing the amine compound with an inorganic nitrite salt to form a diazonium salt.

2. The process of claim 1, which comprises stabilizing the diazonium salt with a complex anion and isolating the stabilized diazonium salt.

3. The process of claim 1, which comprises providing reactive groups, optionally present at the aromatic amide, with protective groups, which are not split off under the conditions of amide hydrolysis.

4. The process of claim 1, wherein the hydrolyzing agent is a mineral acid.

5. The process of claim 1, wherein the hydrolyzing agent is an alcoholic mineral acid, of a C₁-C₄ alcohol.

6. The process of claim, wherein the mineral acid is hydrochloric acid, hydrobromic acid or sulfuric acid.

7. The process of claim 5, wherein the alcohol is methanol, ethanol, n-propanol or isopropanol.

8. The process of claim 1, wherein the amide hydrolysis is carried out at a temperature between 20° C. and 100° C.

9. The process of claim 1, wherein the diazotization step is carried out at a temperature between −10° C. and +10° C.

10. The process of claim 1, wherein the the amide hydrolysis and the diazotization step are carried out within the same reaction mixture and without isolating the amine.

11. The process of claim 1, wherein the diazonium salt is converted into a stable salt and optionally isolated.

12. The process of claim 1 for synthesizing p-benzyloxyphenyl-diazonium tetrafluoroborate, wherein 4-acetamidophenol is converted with benzyl bromide and protected, the amide bond of the product, so obtained, is split selectively with aqueous, alcoholic mineral acid and the mixture, so obtained, is diazotized with an inorganic nitrite salt and, optionally, converting the diazonium salts obtained by treatment with NH₄BF₄ into the tetrafluoroborate salt and optionally isolated.

13. The process of claim 5, wherein the mineral acid is hydrochloric acid, hydrobromic acid or sulfuric acid.

14. The process of claim 6, wherein the alcohol is methanol, ethanol, n-propanol or isopropanol.
